(19) United States
(12) Patent Application Publication
(10) Pub. No.: US 2002/0172627 A1
(43) Pub. Date: Nov. 21, 2002

(54) System for decomposing harmful substances by using photocatalyst

(76) Inventor: Akira Aoyagi, Sakura-shi (JP)

Correspondence Address: BIRCH STEWART KOLASCH & BIRCH, PO BOX 747, FALLS CHURCH, VA, 22040-0747, US

(21) Appl. No.: 09/833,436

(22) Filed: Apr. 13, 2001

(30) Foreign Application Priority Data
Mar. 16, 2001 (JP) 2001-75715

(51) Int. Cl. B01J 19/08

(52) U.S. Cl. 422/186.3; 422/186

(57) ABSTRACT

The present invention relates to a system for decomposing harmful substances by using photocatalyst. The system for decomposing harmful substances by using photocatalyst of the present invention comprises a flow passage where a fluid containing harmful substances flows, a rotator having a photocatalyst fixed on surface thereof and installed in said flow passage in rotatable state, and photo-irradiation means for irradiating light to said photocatalyst. In the system, the photocatalyst fixed on the rotator in rotation is activated by the photo-irradiation means and decomposes harmful substances in the fluid, while the fluid is agitated by the rotation of the rotator.

### BACKGROUND OF THE INVENTION

1. Field of the Invention

The present invention relates to a system for decomposing harmful substances by using photocatalyst.

2. Description of the Prior Art

In recent years, the importance has been increasingly placed on the technique to decompose harmful substances such as dioxin, PCB, etc. contained in waste water, exhaust air, etc. and to turn them into harmless substances or to remove them. Among the techniques used for such purpose, the technique to decompose harmful substances by using photocatalyst is highly evaluated because of its suitability for practical application, and fervent attempts have been made on the research and the development of this technique. A photocatalyst is a catalyst, which is activated and promotes chemical reaction when light is irradiated to it. The method for decomposing harmful substances by using the photocatalyst can be roughly divided to the following two methods: One is a method to fix the photocatalyst and to use it, and the other is a method to mix powder of photocatalyst in a reaction system of gas or liquid and to use it. Among these methods, the method to use by fixing photocatalyst is advantageous in that the reaction system because the photocatalyst can be easily separated after the reaction, and this method is widely adopted for the system for decomposing harmful substances by using photocatalyst.

In a conventional type system for decomposing harmful substances by using photocatalyst, as disclosed in JP-A-10-337579, for instance, water containing harmful substances is passed into a reaction column filled with photocatalyst, and photocatalyst is activated by an ultraviolet light lamp installed at the center of the reaction column to decompose harmful substances. Also, as disclosed in JP-A-09-206558, for instance, ultraviolet ray from an ultraviolet light lamp is irradiated to the photocatalyst supported on a planar support member, and a gas containing harmful substances is brought into contact with the photocatalyst, and the harmful substances are decomposed.

However, in the system for decomposing harmful substances by using the reaction column filled with photocatalyst, the reaction system can be brought into full contact with the photocatalyst, while the photocatalyst layer is thick and the photocatalyst intensely absorbs ultraviolet light. As a result, many parts of the photocatalyst are not completely activated by the ultraviolet ray, and the harmful substances contained in the fluid passing through those parts of the catalyst are not completely decomposed. Also, in the system for decomposing harmful substances by using photocatalyst supported on the support member, most or all of the catalysts are activated by the ultraviolet ray, while the area of ultraviolet light irradiation is not extensive enough and percentage of the fluid passing through the system without being brought into contact with the photocatalysts is increased, and harmful substances are not completely decomposed. On the other hand, in case powder photocatalyst is mixed in the reaction system of gas or liquid and is used, the contact between the catalyst and the reaction substances would be better, while it is difficult to separate the photocatalyst from the reaction system of gas or liquid after the reaction.

### SUMMARY OF THE INVENTION

It is an object of the present invention to provide a system for decomposing harmful substances by using photocatalyst in more efficient manner.

The present invention provides a system for decomposing harmful substances by using photocatalyst where fluid containing harmful substances enters the system through an inlet and is discharged from an outlet of the system, and the system comprises a rotator having a photocatalyst fixed on its surface and installed in a flow passage of the fluid in such manner that it can be rotated, and a light source for irradiating light to the photocatalyst, whereby the fluid in the system is agitated by rotating the rotator while light is irradiated to the photocatalyst, frequency of contact between the harmful substances and the photocatalyst is increased, and the harmful substances contained in the fluid are decomposed at high decomposition ratio.

According to this system for decomposing harmful substances by using the photocatalyst, it is possible to decompose harmful substances in exhaust gas or in waste water at high decomposition ratio and to turn them into harmless substances.

### BRIEF DESCRIPTION OF THE DRAWINGS

FIG. 1 to FIG. 5 each represents a drawing to explain a system for decomposing harmful substances by using photocatalyst according to the present invention.

### DETAILED DESCRIPTION OF THE INVENTION

Description will be given below on a system for decomposing harmful substances using photocatalyst according to the present invention.

[Embodiment 1]

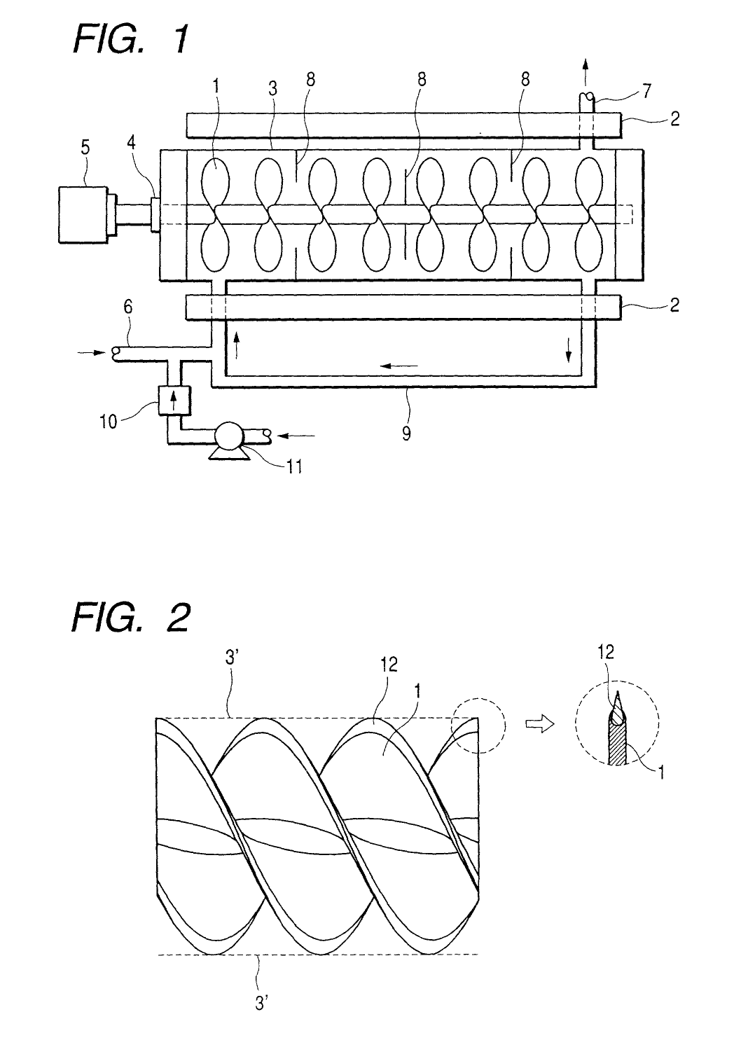

FIG. 1 shows a first embodiment of the present invention. As shown in FIG. 1, the system according to the present invention comprises a rotator 1, a light source 2, a transparent member 3, a seal 4, a motor 5, an inlet for fluid 6, an outlet for fluid 7, baffle plates 8, a circulation passage 9, a check valve 10, and a pump or a fan 11. The transparent member 3 is designed in cylindrical shape, and it also serves as an outer wall of a reaction vessel, in which decomposition of harmful substances by using photocatalyst takes place. The rotator 1 comprises a photocatalyst fixed on its surface, and it is installed in a passage of a fluid in the transparent member 3 in such manner that it can be rotated. A rotating shaft of the rotator 1 is passed via the seal 4 from inside to outside of the transparent member 3. The motor 5 rotates and drives the rotating shaft of the rotator 1. Each of the baffle plates 8 is a disk plate with a concentric hole (cross-section is shown in the figure) to elongate the passage of the fluid in the transparent member 3. The pump or the fan 11 is installed for the purpose of introducing the air or oxygen into the system. The check valve 10 prevents the backward flow of the fluid. In the figure, each arrow indicates flowing direction of the fluid.

In this case, the photo-irradiation means irradiates the light emitted from the light source 2 to the photocatalyst via the transparent member 3.

In this embodiment, the rotator 1 comprises a plurality of rotary vanes each resembling to a propeller of ship and fixed on a rotating shaft, and the rotator 1 is rotated by driving force of the motor 5 via the seal 4.

The light source 2 is an ultraviolet light source such as a mercury lamp or a visible light source such as a fluorescent lamp. The transparent member 3 is a cylinder made of fused silica, for instance, when the ultraviolet light source is used as the light source 2. The transparent member 3 is a colorless transparent glass cylinder, for instance, when the visible light source is used as the light source 2. Titanium dioxide is used as the photocatalyst, for instance. In case visible light source is used as the light source 2, it is preferable to use gas-plasma-treated titanium dioxide.

A light beam emitted from the light source 2 passes through the transparent member 3 and enters the photocatalyst on the surface of the rotary vanes. The fluid containing harmful substances enters an inlet for fluid 6, passes through the cylindrical transparent member 3 and goes out of the outlet 7. When the fluid passes through the cylindrical transparent member 3, the fluid is agitated by the rotation of the rotator 1. The harmful substances contained in the fluid are brought into contact with the photocatalyst activated by photo-irradiation and are decomposed. Because the fluid is agitated by the rotation of the rotator 1, the frequency of the contact between the harmful substances and the photocatalyst is increased. As a result, the decomposition ratio of the harmful substances is increased. By the baffle plates 8 installed in the passage of the fluid, the passage of the fluid is elongated, and this further increases the frequency of contact between the harmful substances and the photocatalyst. Hence, it increases the decomposition ratio of the harmful substances.

In the present embodiment, a circulation passage 9 for the fluid is provided. A force to push the fluid toward the outlet 7 is provided by the rotation of the rotary vanes, which are component parts of the rotator 1. As a result, difference occurs in the pressure of the fluid in the circulation passage 9. By this pressure difference, the fluid is sent in the direction of the arrow as shown in FIG. 1 through the circulation passage 9 and it is circulated in the system. Then, the fluid passes through points where the photocatalyst is activated by the photo-irradiation as many times as more than one time, and this contributes to the increase of the decomposition ratio of the harmful substances. By increasing the rotating speed of the rotator 1, the number of times of circulation of the fluid can be increased, and the fluid in the system can be turned to the state of turbulent flow. This further increases the frequency of the contact between the harmful substances and the photocatalyst. Hence, it increases the decomposition ratio of the harmful substances.

The decomposition of harmful substances—above all, the decomposition of organic harmful substances—is enhanced due to the presence of oxygen. Sending the air or oxygen into the passage of the fluid via the check valve 10 using the pump or the fan 11 permits the increased efficiency of the decomposition of the harmful substances.

FIG. 2 shows a variation of the rotator 1 different from the one shown in FIG. 1. As shown in FIG. 2, the rotator 1 comprises rotary vanes designed in triple spiral shape fixed on a rotating shaft. A photocatalyst is fixed on the surface of each of the rotary vanes. Further, a flexible thin plate 12 made of polymeric fluorocarbon resin, for instance, is provided on edge of the rotary vane. This thin plate 12 is designed to be thinner toward outer edge, and the thinnest outer edge is in contact with inner surface 3′ of the transparent member 3. When the rotator 1 is rotated, it rubs the inner surface 3′ of the transparent member 3 and has a function to remove stains and dirt attached on the surface 3′. Specifically, by using the rotator 1 with such features, stains and dirt on portions of the transparent member 3 in contact with the fluid are removed during the operation of the system, and photo-irradiation efficiency to the photocatalyst can be maintained. Because the light source 2 is provided outside the transparent member 3, air cooling or liquid cooling of the light source 2 can be performed easily, and this also facilitates the replacement of the light source 2.

(An Example of the System Operation)

Applying a system for decomposing harmful substances by using photocatalyst as shown in FIG. 1, harmful substances were decomposed. The results of the operation is described below:

As the light source 2, four ultraviolet light lamps of 20 W each were used. Most of the ultraviolet ray emitted from the light source 2 was a mercury line with wavelength of 253.7 nm, and the radiation intensity of the line on the lamp surface was 8.5 mW/cm ². The transparent member 3, which serves as reaction vessel was made of fused silica, and it was 31.5 mm in inner diameter and 500 mm in length. Using a system with the above arrangement and features, decomposing reaction was performed one time each in the case where the fluid is gas and the case where it is liquid.

(In case the Fluid is Air, i.e. Gas)

There were 27 rotary vanes in the rotator 1, and the surface area of each vane on front surface and on rear surface together was 8 cm². On the surface, titanium dioxide (ST-K03; manufactured by Ishihara Techno Co., Ltd.) was coated as photocatalyst. The rotating speed of the rotary vane was set to 1500 rpm, and flow rate of the air at the inlet 6 and the outlet 7 was set to 150 cm³/min.

The air containing very small quantity of monochlorobenzene was used as an example of harmful substance, and the decomposing reaction was performed for about 2 minutes under the above conditions. Gas detection tube method, where the concentration of the component to be detected is measured by the length of color-changed zone in the tube, was used for the analysis of very small quantities of the components contained in the air. The results are summarized in Table 1.

TABLE 1

| | | Decomposed gas | |
|---|---|---|---|
| | | Rotary | Rotary |
| | Original | vanes | vanes |
| Components | gas | not rotated | rotated |
| Monochlorobenzene | 10 ppm | 6 ppm | 0 ppm |
| Carbon dioxide | 25 ppm | 50 ppm | 100 ppm |
| Chloride (including HCl) | 0 ppm | 0 ppm | 10 ppm |
| Chlorine gas | 0 ppm | 0 ppm | 0 ppm |
| Phosgene | 0 ppm | 0 ppm | 0 ppm |

As shown in Table 1, when the rotary vanes coated with photocatalyst were not rotated (this corresponds to the prior art), the quantity of monochlorobenzene decreased only from 10 to 6 ppm. In contrast, when the rotary vanes were rotated, the content of monochlorobenzene decreased to such amount that it cannot be detected by the detection tube. As a result, it was confirmed that the present invention has an effect to extensively increase the decomposition ratio of harmful substances by rotating the rotator 1 having the photocatalyst fixed on its surface compared with the prior art.

(In Case the Fluid is Water, i.e. Liquid)

There were 17 rotary vanes in the rotator 1, and the surface area of each vane on front surface and on rear surface together was 8 cm². On its surface, titanium dioxide (ST-K03; manufactured by Ishihara Techno Co., Ltd.) was coated as photocatalyst. The rotating speed of the rotary vanes was set to about 600 rpm, and the quantity of the fluid processed at one time was set to 250cm³. Decomposing reaction was performed by varying the reaction time. Also, decomposing reaction was performed by mixing oxygen gas into the fluid at a rate of 30 cm³/min.

In pure water with electric conductivity of 0 μS, oxalic acid was dissolved as an example of harmful substance at the concentration of 10⁻⁴ mole/liter, and the decomposing reaction was performed under the above conditions. The decrease of oxalic acid concentration was detected by the decrease of electric conductivity of the liquid. The results are summarized in Table 2.

TABLE 2

| | Electric conductivity (μS) | | |
|---|---|---|---|
| | Rotary vanes | Rotary vanes | Rotary vanes |
| Reaction | rotated; | rotated; | not rotated; |
| time (min) | air mixed | air not mixed | air not mixed |
| 0 | 70 | 70 | 70 |
| 3 | 0 | 30 | 40 |
| 6 | 0 | 0 | 10 |
| 9 | 0 | 0 | 0 |

As shown in Table 2, decomposition of oxalic acid was most efficiently performed in the case where rotary vanes were rotated and the air was mixed. The second most efficient case was the case where rotary vanes were rotated but the air was not mixed. In the case where the rotary vanes were not rotated and the air was not mixed, the decomposing reaction occurred most inefficiently. These results confirmed that decomposition ratio of harmful substances can be increased by rotating the rotator 1 with photocatalyst fixed on its surface, and also that the effect can be further enhanced when the air is mixed.

In the above embodiment, monochlorobenzene and oxalic acid were used as examples of the harmful substances. Because the photocatalyst activated by ultraviolet light enhances decomposing reaction of a wide variety of substances, similar effects can be obtained in the case where harmful substances are dioxin, PCB, etc.

[Embodiment 2]

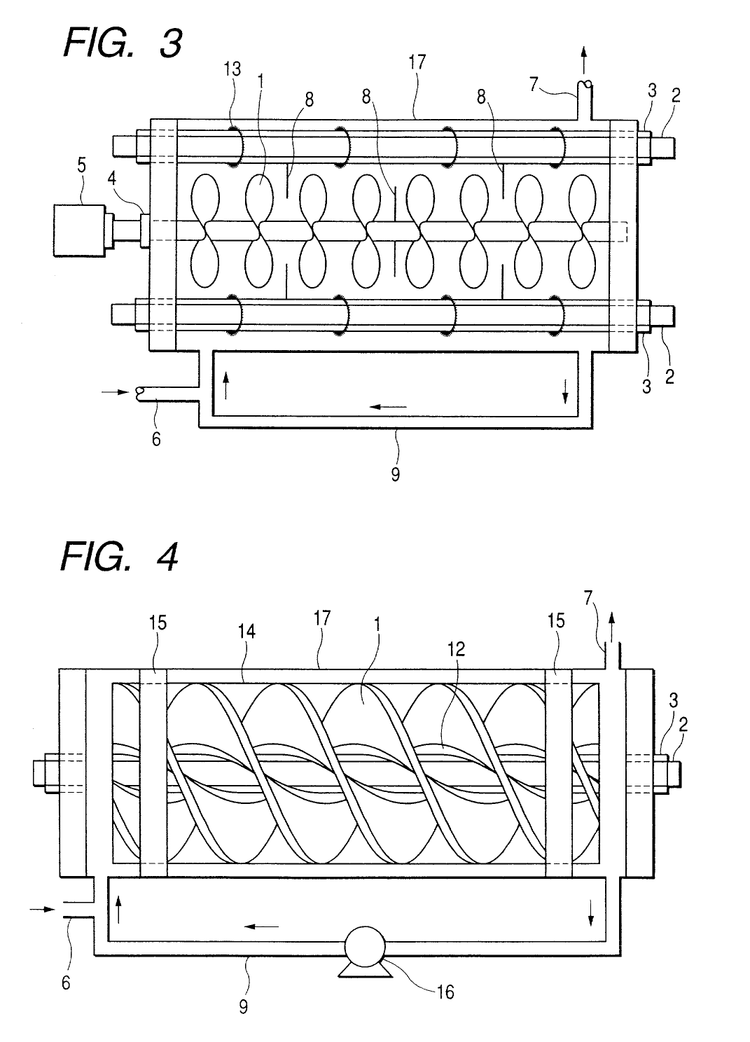

A second embodiment of the present invention is shown in FIG. 3. As shown in FIG. 3, the system comprises a rotator 1, a light source 2, a transparent member 3, a seal 4, a motor 5, an inlet for fluid 6, an outlet for fluid 7, baffle plates 8, a circulation passage 9, annular brushes 13, and a reaction vessel 17.

Decomposing reaction of harmful substances by photocatalyst takes place inside the reaction vessel 17. The rotator 1 has a photocatalyst fixed on its surface, and it is installed in a flow passage of the fluid in the reaction vessel 17 in such manner that it can be rotated. The light source 2 emits the light to irradiate the photocatalyst. The transparent member 3 is installed as an outer tube of the light source 2. The seal 4 allows the rotating shaft of the rotator 1 to pass through from inside to outside of the reaction vessel 17. The motor 5 rotates and drives the rotating shaft of the rotator 1. Each of the baffle plates 8 is a disk plate with a concentric hole (cross-section is shown in the figure) to elongate flow passage of the flow in the transparent member 3. Each arrow in the figure indicates flowing direction of the fluid.

In this case, the photo-irradiation means irradiates the light emitted from the light source 2 to the photocatalyst via the transparent member 3.

The light source 2 is an ultraviolet light source such as a mercury lamp or a visible light source such as a fluorescent lamp. The transparent member 3 is a cylinder made of fused silica, for instance, when the ultraviolet light source is used as the light source 2. The transparent member 3 is a colorless transparent glass cylinder, for instance, when the visible light source is used as the light source 2. Titanium dioxide is used as the photocatalyst, for instance. In case visible light source is used as the light source 2, it is preferable to use gas-plasma-treated titanium dioxide.

The difference between the present embodiment from the first embodiment is that most or all of the light emitting portions of the light source 2 are surrounded by the fluid. The effect of the present embodiment to increase decomposition ratio of harmful substances by rotation of the rotator 1 is similar to the effect of the first embodiment. However, in the present embodiment, annular brushes 13 with each brush comprising a bar-like brush component with its both ends connected to each other to have an annular shape is provided as shown in FIG. 3 and this is provided outside of the tubular transparent member 3. By the driving force caused from the movement of the fluid, the annular brushes 13 are rotated outside the transparent member 3. These brushes 13 remove stains and dirt on outer surface of the transparent member 3 and maintain irradiation efficiency of the light. The light source 2 is separated from the flow passage of the fluid by the transparent member 3, and this facilitates air cooling or liquid cooling of the light source 2. To replace the light source 2, there is no need to replace and demount the members, which are used to form the flow passage, and the light source 2 can be replaced easily.

[Embodiment 3]

FIG. 4 shows a third embodiment of the present invention. As shown in FIG. 4, the system comprises a rotator 1, a light source 2, a transparent member 3, an inlet for fluid 6, an outlet for fluid 7, a circulation passage 9, a flexible thin plate 12, a cylinder 14, bearings 15, a pump or a fan 16, and a reaction vessel 17.

Decomposing reaction of the harmful substances by using photocatalyst takes place inside the reaction vessel 17. The rotator 1 has photocatalyst fixed on its surface and is installed in the flow passage of the fluid in the reaction vessel 17 in such manner that it can be rotated. The light source 2 emits the light to irradiate the photocatalyst. The transparent member 3 is designed in cylindrical shape and it is installed as an outer tube of the light source 2. The flexible thin plate 12 is mounted on the rotator 1. The cylinder 14 has the rotator 1 inside and it is installed in the reaction vessel 17. The bearings 15 retain the cylinder 14 in rotatable state. The pump or the fan 16 circulates the fluid inside the system. In the figure, each arrow indicates flowing direction of the fluid.

In this case, the photo-irradiation means irradiates the light emitted from the light source 2 to the photocatalyst via the transparent member 3.

In the present embodiment, the cylinder 14 is retained in rotatable state within the reaction vessel 17 by the bearings 15. The rotator 1 designed in triple spiral shape is fixed inside the cylinder 14 and it is rotated together with the cylinder 14. In FIG. 4, internal portion of the cylinder 14 is also shown. The cylindrical transparent member 3 is installed in the fluid so that it is coaxial with the rotating shaft of the rotor 1, and the light source 2 is installed in it.

The difference of the present embodiment from the first and the second embodiments is that the rotator 1 is not rotated on the rotating shaft, but it is rotated by the driving force caused by the movement of the fluid inside the reaction vessel 17. As a result, there is no need to provide the seal (e.g. the seal shown by 4 in FIG. 1), which allows the rotating shaft to pass from outside to inside. This eliminates the problems caused by the seal such as the need to repair the seal.

In the present embodiment, the light source 2 is surrounded by the rotator 1, and most of light beams emitted from the light source 2 are irradiated to the photocatalyst fixed on the surface of the rotator 1, and this means that utilization ratio of the light is increased. If photocatalyst is also fixed on inner surface of the cylinder 14, utilization ratio of the light is further increased.

On inner edge of the rotator 1, the flexible thin plate 12 is provided. Like the case of the first and the second embodiments using the rotator 1, the thin plate 12 rubs outer surface of the transparent member 3 as the rotator 1 is rotated, and this contributes to the removal of stains and dirt on the surface. This is also helpful for the maintenance of the photo-irradiation efficiency to the photocatalyst.

In the present embodiment, in addition to the rotating driving force caused by the inflow of the fluid into the system, the pump or the fan 16 is operated, and the rotator 1 is rotated by rotating and driving force generated by the circulation of the fluid. As a result, it is possible to improve decomposition ratio of the harmful substances. By increasing the propelling force of the pump or the fan 16, flow velocity of the fluid can be increased and circulation efficiency of the fluid can be raised. The fluid in the system can be turned to the state of turbulent flow. This further increases the frequency of contact between the harmful substances and the photocatalyst and to increase decomposition ratio of the harmful substances.

Because the light source 2 is separated from the flow passage of the fluid by the transparent member 3, it is easier to perform air cooling or liquid cooling of the light source 2. To replace the light source 2, there is no need to replace and demount the members, which are used to form the flow passage, and the light source 2 can be easily replaced.

In the present embodiment, the cylinder 14 is provided with the rotator 1 inside, while a cylinder 14 with multiple small bores may be used as the cylinder 14, or the cylinder 14 may be fabricated from a screen designed in cylindrical shape.

[Embodiment 4]

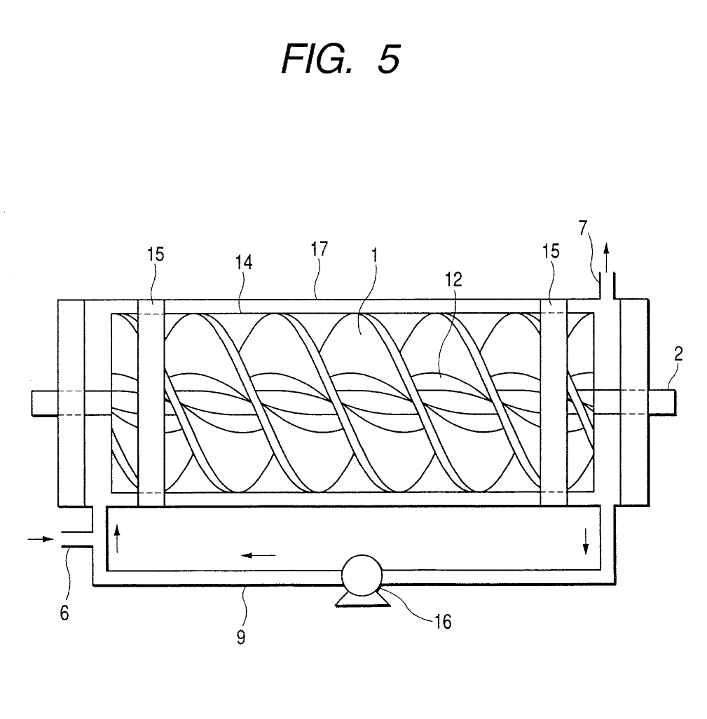

A fourth embodiment of the present invention is shown in FIG. 5. As shown in FIG. 5, the system comprises a rotator 1, a light source 2, an inlet for fluid 6, an outlet for fluid 7, a circulation passage 9, a flexible thin plate 12, a cylinder 14, bearings 15, a pump or a fan 16, and a reaction vessel 17.

Decomposing reaction of harmful substances by using photocatalyst takes place in the reaction vessel 17. The rotator 1 has a photocatalyst fixed on its surface and it is installed in the flow passage of the fluid in the reaction vessel 17 in rotatable state. The light source 2 emits the light to irradiate the photocatalyst. The cylinder 14 has the rotator 1 inside, and it is installed in the reaction vessel 17. The bearings 15 retain the cylinder 14 in rotatable state. The pump or the fan 16 drives the fluid to circulate within the system. Each arrow in the figure indicates flowing direction of the fluid.

In this case, the photo-irradiation means exposes outer wall of the light source 2 into the flow passage and irradiates the light emitted from the light source 2 to the photocatalyst through the outer wall.

The present embodiment is different from the third embodiment in that the transparent member is not used, and that outer wall of the light source 2 comes into direct contact with the fluid. This further increases utilization ratio of the light emitted from the light source 2.

The other arrangement and features are the same as those of the third embodiment. By the present embodiment, it is possible to decompose harmful substances at high ratio as in the case of the third embodiment.

In the first and the second embodiments, it may be designed in such manner that a pump or a fan as used in the third or the fourth embodiment may be installed to speed up the circulation of the fluid in the system.

Also, in the second, the third, or the fourth embodiment, a pump or a fan to mix the air or oxygen with the fluid and a check valve to prevent the backward flow of the fluid may be installed as in the first embodiment with the purpose of promoting the decomposition of harmful substance.

And also, in the case where the fluid containing harmful substances need to be treated in a high rate by the system according to present invention, the fluid is preferably treated by parallel operation using a plurality of reaction vessels. Furthermore, in the system where the light source is placed outside the reaction vessel, as in the first embodiment, the light source is preferably surrounded by the vessels so as to utilize the light emitted from the light source more effectively.

What is claimed is:

1. A system for decomposing harmful substances by using photocatalyst, comprising a flow passage where a fluid containing at least one kind of harmful substance flows, a rotator having a photocatalyst fixed on surface thereof and installed in said flow passage in rotatable state, and photo-irradiation means for irradiating light to said photocatalyst.

2. A system for decomposing harmful substances by using photocatalyst according to claim 1, wherein outer wall of a light source is exposed to said flow passage and said photo-irradiation means is means for irradiating the light emitted from said light source toward said photocatalyst through said outer wall.

3. A system for decomposing harmful substances by using photocatalyst according to claim 1, wherein said photo-irradiation means is means for irradiating the light emitted from a light source toward said photocatalyst via a transparent member.

4. A system for decomposing harmful substances by using photocatalyst according to claim 2, wherein outer wall of said light source is provided with means for removing stains and dirt on a portion in contact with said fluid during operation of said system.

5. A system for decomposing harmful substances by using photocatalyst according to claim 3, wherein said transparent member is provided with means for removing stains and dirt on a portion in contact with said fluid during operation of said system.

6. A system for decomposing harmful substances by using photocatalyst according to claim 1, wherein there is provided a circulation passage for circulating said fluid within said system.

7. A system for decomposing harmful substances by using photocatalyst according to claim 2, wherein there is provided a circulation passage for circulating said fluid within said system.

8. A system for decomposing harmful substances by using photocatalyst according to claim 3, wherein there is provided a circulation passage for circulating said fluid within said system.

9. A system for decomposing harmful substances by using photocatalyst according to claim 4, wherein there is provided a circulation passage for circulating said fluid within said system.

10. A system for decomposing harmful substances by using photocatalyst according to claim 5, wherein there is provided a circulation passage for circulating said fluid within said system.

11. A system for decomposing harmful substances by using photocatalyst according to claim 6, wherein there is provided a pump or a fan within said circulation passage.

12. A system for decomposing harmful substances by using photocatalyst according to claim 7, wherein there is provided a pump or a fan within said circulation passage.

13. A system for decomposing harmful substances by using photocatalyst according to claim 8, wherein there is provided a pump or a fan within said circulation passage.

14. A system for decomposing harmful substances by using photocatalyst according to claim 9, wherein there is provided a pump or a fan within said circulation passage.

15. A system for decomposing harmful substances by using photocatalyst according to claim 10, wherein there is provided a pump or a fan within said circulation passage.

16. A system for decomposing harmful substances by using photocatalyst according to claim 1, wherein said system has means for mixing air or oxygen with said fluid.

17. A system for decomposing harmful substances by using photocatalyst according to claim 2, wherein said system has means for mixing air or oxygen with said fluid.

18. A system for decomposing harmful substances by using photocatalyst according to claim 3, wherein said system has means for mixing air or oxygen with said fluid.

19. A system for decomposing harmful substances by using photocatalyst according to claim 4, wherein said system has means for mixing air or oxygen with said fluid.

20. A system for decomposing harmful substances by using photocatalyst according to claim 5, wherein said system has means for mixing air or oxygen with said fluid.

21. A system for decomposing harmful substances by using photocatalyst according to claim 6, wherein said system has means for mixing air or oxygen with said fluid.

22. A system for decomposing harmful substances by using photocatalyst according to claim 7, wherein said system has means for mixing air or oxygen with said fluid.

23. A system for decomposing harmful substances by using photocatalyst according to claim 8, wherein said system has means for mixing air or oxygen with said fluid.

24. A system for decomposing harmful substances by using photocatalyst according to claim 9, wherein said system has means for mixing air or oxygen with said fluid.

25. A system for decomposing harmful substances by using photocatalyst according to claim 10, wherein said system has means for mixing air or oxygen with said fluid.

26. A system for decomposing harmful substances by using photocatalyst according to claim 11, wherein said system has means for mixing air or oxygen with said fluid.

27. A system for decomposing harmful substances by using photocatalyst according to claim 12, wherein said system has means for mixing air or oxygen with said fluid.

28. A system for decomposing harmful substances by using photocatalyst according to claim 13, wherein said system has means for mixing air or oxygen with said fluid.

29. A system for decomposing harmful substances by using photocatalyst according to claim 14, wherein said system has means for mixing air or oxygen with said fluid.

30. A system for decomposing harmful substances by using photocatalyst according to claim 15, wherein said system has means for mixing air or oxygen with said fluid.

31. A system for decomposing harmful substances by using photocatalyst according to claim 1, wherein said photo-irradiation means utilizes an ultraviolet light source.

32. A system for decomposing harmful substances by using photocatalyst according to claim 2, wherein said light source is an ultraviolet light source.

33. A system for decomposing harmful substances by using photocatalyst according to claim 3, wherein said light source is an ultraviolet light source.

34. A system for decomposing harmful substances by using photocatalyst according to claim 4, wherein said light source is an ultraviolet light source.

35. A system for decomposing harmful substances by using photocatalyst according to claim 5, wherein said light source is an ultraviolet light source.

36. A system for decomposing harmful substances by using photocatalyst according to claim 6, wherein said photo-irradiation means utilizes an ultraviolet light source.

37. A system for decomposing harmful substances by using photocatalyst according to claim 7, wherein said light source is an ultraviolet light source.

38. A system for decomposing harmful substances by using photocatalyst according to claim 8, wherein said light source is an ultraviolet light source.

39. A system for decomposing harmful substances by using photocatalyst according to claim 9, wherein said light source is an ultraviolet light source.

40. A system for decomposing harmful substances by using photocatalyst according to claim 10, wherein said light source is an ultraviolet light source.

41. A system for decomposing harmful substances by using photocatalyst according to claim 11, wherein said photo-irradiation means utilizes an ultraviolet light source.

42. A system for decomposing harmful substances by using photocatalyst according to claim 12, wherein said light source is an ultraviolet light source.

43. A system for decomposing harmful substances by using photocatalyst according to claim 13, wherein said light source is an ultraviolet light source.

44. A system for decomposing harmful substances by using photocatalyst according to claim 14, wherein said light source is an ultraviolet light source.

45. A system for decomposing harmful substances by using photocatalyst according to claim 15, wherein said light source is an ultraviolet light source.

46. A system for decomposing harmful substances by using photocatalyst according to claim 16, wherein said photo-irradiation means utilizes an ultraviolet light source.

47. A system for decomposing harmful substances by using photocatalyst according to claim 17, wherein said light source is an ultraviolet light source.

48. A system for decomposing harmful substances by using photocatalyst according to claim 18, wherein said light source is an ultraviolet light source.

49. A system for decomposing harmful substances by using photocatalyst according to claim 19, wherein said light source is an ultraviolet light source.

50. A system for decomposing harmful substances by using photocatalyst according to claim 20, wherein said light source is an ultraviolet light source.

51. A system for decomposing harmful substances by using photocatalyst according to claim 21, wherein said photo-irradiation means utilizes an ultraviolet light source.

52. A system for decomposing harmful substances by using photocatalyst according to claim 22, wherein said light source is an ultraviolet light source.

53. A system for decomposing harmful substances by using photocatalyst according to claim 23, wherein said light source is an ultraviolet light source.

54. A system for decomposing harmful substances by using photocatalyst according to claim 24, wherein said light source is an ultraviolet light source.

55. A system for decomposing harmful substances by using photocatalyst according to claim 25, wherein said light source is an ultraviolet light source.

56. A system for decomposing harmful substances by using photocatalyst according to claim 26, wherein said photo-irradiation means utilizes an ultraviolet light source.

57. A system for decomposing harmful substances by using photocatalyst according to claim 27, wherein said light source is an ultraviolet light source.

58. A system for decomposing harmful substances by using photocatalyst according to claim 28, wherein said light source is an ultraviolet light source.

59. A system for decomposing harmful substances by using photocatalyst according to claim 29, wherein said light source is an ultraviolet light source.

60. A system for decomposing harmful substances by using photocatalyst according to claim 30, wherein said light source is an ultraviolet light source.

61. A system for decomposing harmful substances by using photocatalyst according to claim 1, wherein said photo-irradiation means utilizes a visible light source.

62. A system for decomposing harmful substances by using photocatalyst according to claim 2, wherein said light source is a visible light source.

63. A system for decomposing harmful substances by using photocatalyst according to claim 3, wherein said light source is a visible light source.

64. A system for decomposing harmful substances by using photocatalyst according to claim 4, wherein said light source is a visible light source.

65. A system for decomposing harmful substances by using photocatalyst according to claim 5, wherein said light source is a visible light source.

66. A system for decomposing harmful substances by using photocatalyst according to claim 6, wherein said photo-irradiation means utilizes a visible light source.

67. A system for decomposing harmful substances by using photocatalyst according to claim 7, wherein said light source is a visible light source.

68. A system for decomposing harmful substances by using photocatalyst according to claim 8, wherein said light source is a visible light source.

69. A system for decomposing harmful substances by using photocatalyst according to claim 9, wherein said light source is a visible light source.

70. A system for decomposing harmful substances by using photocatalyst according to claim 10, wherein said light source is a visible light source.

71. A system for decomposing harmful substances by using photocatalyst according to claim 11, wherein said photo-irradiation means utilizes a visible light source.

72. A system for decomposing harmful substances by using photocatalyst according to claim 12, wherein said light source is a visible light source.

73. A system for decomposing harmful substances by using photocatalyst according to claim 13, wherein said light source is a visible light source.

74. A system for decomposing harmful substances by using photocatalyst according to claim 14, wherein said light source is a visible light source.

75. A system for decomposing harmful substances by using photocatalyst according to claim 15, wherein said light source is a visible light source.

76. A system for decomposing harmful substances by using photocatalyst according to claim 16, wherein said photo-irradiation means utilizes a visible light source.

77. A system for decomposing harmful substances by using photocatalyst according to claim 17, wherein said light source is a visible light source.

78. A system for decomposing harmful substances by using photocatalyst according to claim 18, wherein said light source is a visible light source.

79. A system for decomposing harmful substances by using photocatalyst according to claim 19, wherein said light source is a visible light source.

80. A system for decomposing harmful substances by using photocatalyst according to claim 20, wherein said light source is a visible light source.

81. A system for decomposing harmful substances by using photocatalyst according to claim 21, wherein said photo-irradiation means utilizes a visible light source.

82. A system for decomposing harmful substances by using photocatalyst according to claim 22, wherein said light source is a visible light source.

83. A system for decomposing harmful substances by using photocatalyst according to claim 23, wherein said light source is a visible light source.

84. A system for decomposing harmful substances by using photocatalyst according to claim 24, wherein said light source is a visible light source.

85. A system for decomposing harmful substances by using photocatalyst according to claim 25, wherein said light source is a visible light source.

86. A system for decomposing harmful substances by using photocatalyst according to claim 26, wherein said photo-irradiation means utilizes a visible light source.

87. A system for decomposing harmful substances by using photocatalyst according to claim 27, wherein said light source is a visible light source.

88. A system for decomposing harmful substances by using photocatalyst according to claim 28, wherein said light source is a visible light source.

89. A system for decomposing harmful substances by using photocatalyst according to claim 29, wherein said light source is a visible light source.

90. A system for decomposing harmful substances by using photocatalyst according to claim 30, wherein said light source is a visible light source.
